(19) United States
(12) Patent Application Publication
(10) Pub. No.: US 2022/0371929 A1
(43) Pub. Date: Nov. 24, 2022

(54) STRUCTURAL ARRANGEMENT APPLIED TO SUPPORTING MEANS FOR WASTEWATER TREATMENT PLANTS

(71) Applicants: Flávio Fernandes SISTI, 1259, Centro, Londrina (BR); Paulo Henrique SCHEIDEMANTEL, Santa Catarina (BR)

(72) Inventors: Flávio Fernandes SISTI, Paraná (BR); Paulo Henrique SCHEIDEMANTEL, Santa Catarina (BR)

(21) Appl. No.: 17/762,183

(22) PCT Filed: Sep. 18, 2020
(86) PCT No.: PCT/BR2020/050371

§ 371 Date: Mar. 21, 2022

(30) Foreign Application Priority Data
Sep. 19, 2019 (BR) 20 2019 019602 0

(51) Int. Cl. C02F 3/10; C12M 1/12; C02F 3/08

(52) U.S. Cl. CPC C02F 3/109; C12M 25/14; C02F 3/08

(57) ABSTRACT

The present utility model relates to supporting means (100) for wastewater treatment plants comprising an outer ring (200), a first inner ring (300), a second inner ring (400) and a third inner ring (500), in which the outer ring (200) is a cylindrical element of diameter (d), height (h) and thickness (e), that has an annular cross section and that delimits the network structure formed therein by inner rings (300, 400, 500), which form a set of openings and respective channels (210, 310, 320, 410, 510).

### FIELD OF APPLICATION

The present utility model pertains to the field of chemical engineering, notably the biological treatment of water, wastewater, sewage, or sludges, in particular biological treatment using moving contact bodies.

### INTRODUCTION

The present utility model relates to a supporting means or biomedia or simply media for use in biological wastewater treatment plants, provided with a body in controlled density, with a specific shape to allow for greater hydrodynamics and surface area, in order to fix, develop and maintain microorganisms responsible for biological treatment.

### STATE OF THE ART

The biomedias treated herein are single components used in biological reactors of wastewater treatment plants, with the objective of providing both surface area and ideal conditions for bacteria responsible for decomposing organic matter to establish and develop.

Biomedias carry high complexity and their development is guided by several variables such as, e.g., hydrodynamics, material technology, potential for fixing the bacterial biofilm to the component, inner dimensions, shear stress, among others.

This complexity has led to highly elaborate solutions but inappropriate to the conditions of tropical countries and also expensive for large-scale use.

An example of biomedia in the state of the art is described by patent document number U.S. Pat. No. 5,981,272, which discloses a novel composite material for application in biological processes, combining a base material and small particles applied over the base using a layer of adhesive. These particles increase the roughness and surface area of the base material, providing valleys and crevices that increase the adherence of bacteria. However, the biomedia from U.S. Pat. No. 5,981,272 are costly to manufacture, both due to the component shape and the need to use adhesives in the component. Furthermore, the geometric features, the hydrodynamic ones in particular, create high friction between the biomedia and the environment, which is inappropriate for fixing the types of bacteria having a more accelerated metabolism.

Further examples of sophisticated solutions that are costly to manufacture and inappropriate for fixing bacteria with faster metabolisms are those disclosed by DE102018100337, FI8703121, CN2846407U, among others.

On the other hand, the fact that they are typically small and apparently simple components has favored the rise of several manufacturers and suppliers of such elements with a lack of technical experience and investment in research and development, resulting in inefficient products with no commitment to technical quality.

An example from the state of the art demonstrating this disregard for larger technical issues regarding biomedia can be found in KR810002068, which discloses a rectangular-shaped biomedia provided four inner longitudinal compartments and four outer fins. This type of solution, although relatively suitable for large-scale manufacturing processes, does not account for the impact of hydrodynamics, shear stresses, among other minimum necessary constructive aspects. This directly affects the biomass performance, directly resulting in losses of treatment efficiency and the aeration system, considerably increasing the operating costs of the treatment plant.

Other examples of solutions are presented in patent documents such as CN105819565, U.S. Pat. No. 6,916,421, CN202594845, among others, that, although of relative low-cost, do not present adequate constructive aspects for good biomass performance.

In general, the dimensions, geometry, and material density of prior-art biomedia have a relation which does not provide ideal conditions for movement and shearing between the components, preventing the continuous biofilm renewal, oxygen exchanges, and thus biomass dynamic development, which additionally increases energy consumption due to the need for more aerators or other movement devices.

Also noted in biomedia in the state of the art is that (in more advanced media) its dimensions and shapes aim to solely meet the requirements of biological treatment, completely neglecting operational aspects, with no consideration for cleaning procedures of media retention, aeration system effects and the like.

Therefore, there is room for an improved structural arrangement applied to supporting means capable of overcoming the deficiencies of the state of the art and providing a functional improvement both in use and manufacture of biomedia of such nature.

### OBJECT OF THE UTILITY MODEL

The object of the present utility model to provide a constructive arrangement in accordance with the features of independent claim 1 of the appended set of claims.

### BRIEF DESCRIPTION OF THE FIGURES

For a better understanding and visualization of the subject matter of the present utility model, it will now be described with reference to the appended figures, representing the obtained functional improvement, in which, schematically:

FIG. 1: shows a top view of a supporting means according to the utility model;

FIG. 2: shows a side view of FIG. 1;

FIG. 3: shows an enlarged view of detail A from FIG. 1;

FIG. 4: shows an enlarged view of detail B from FIG. 1;

FIG. 5: shows a perspective view of FIG. 1;

FIG. 6: shows a perspective view of an oblique supporting means according to the utility model; and

FIG. 7: shows a side view of FIG. 6.

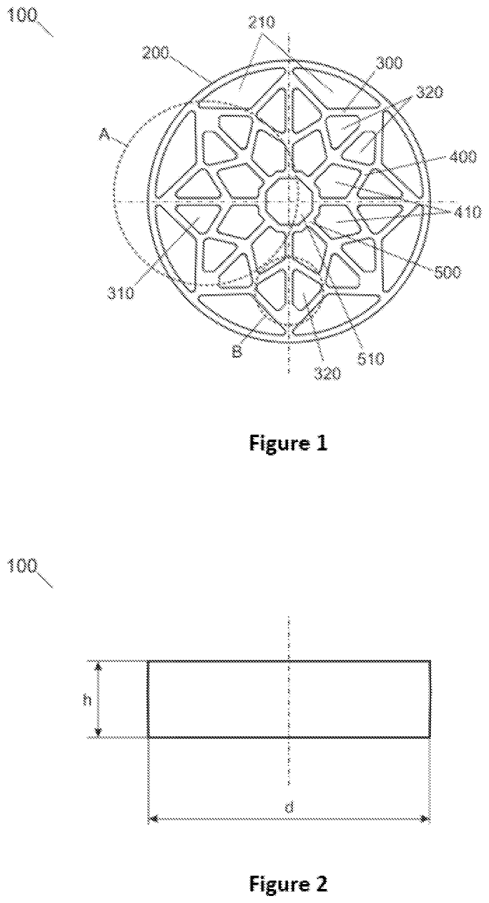

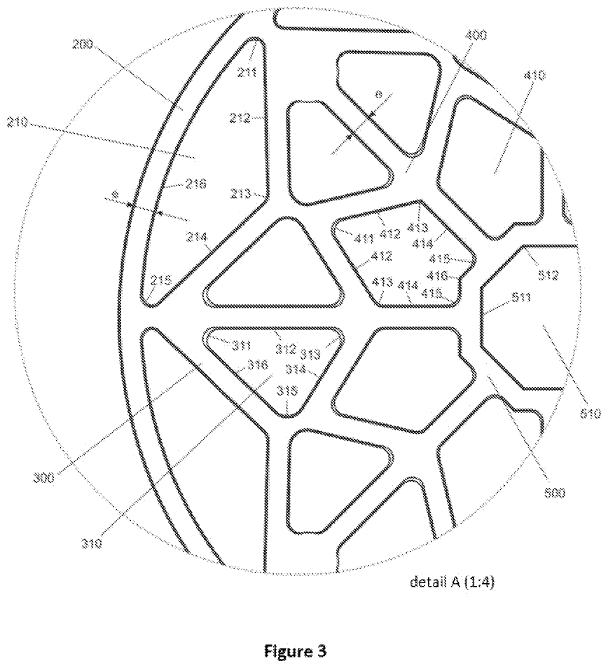

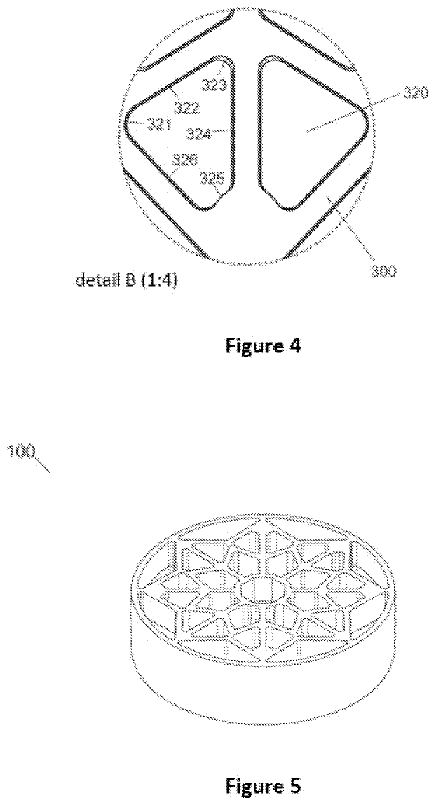

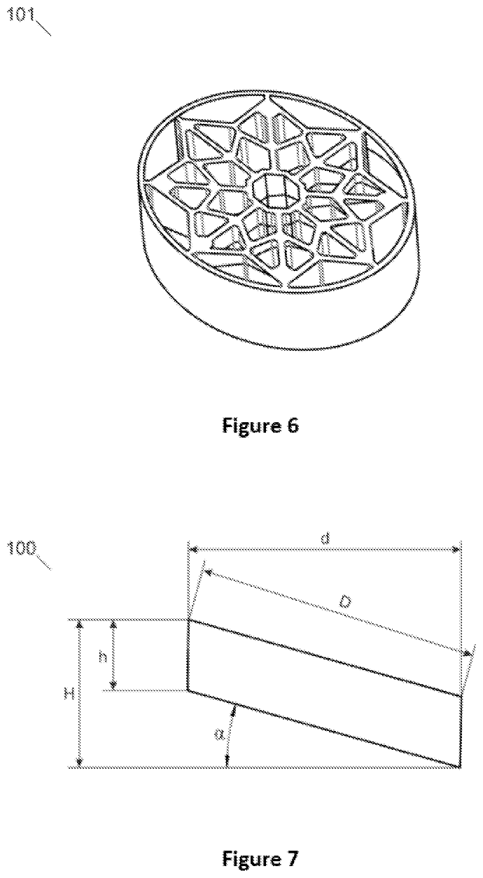

### DETAILED DESCRIPTION OF THE UTILITY MODEL

The appended figures show a supporting means (100) according to the utility model, which comprises:

(i) An outer ring (200);

(ii) A first inner ring (300);

(iii) A second inner ring (400); and

(iv) A third inner ring (500).

The outer ring (200) is a cylindrical element of diameter (d), height (h), and thickness (e), that has annular cross section and which delimits the network structure formed therein.

The dimensions of the supporting means (100) according to the invention will range according to the application and need.

However, there are ideal proportions between measurements which provide the best results.

The ratio between diameter (d) and height (h) ranges between 1 and 5 times, preferably a ratio of 3.7, i.e., diameter (d) is 3.7 times greater than the height (h).

The ideal measurement ranges comprise, for example, a diameter (d) ranging between 10 and 100 mm, preferably 37 mm, whereas the height (h) ranges between 1 and 50 mm, preferably 10 mm, keeping the ratio of 3.7 for the optimal case.

The thickness (e) will range especially according to the dimensions of height (h) and diameter (d) of the material to be used in the manufacturing process, the type of reactor and others, ranging, for the example above, between 0, 1 and 5 mm, preferably being between 0.6 and 0.9 mm.

The shape aims to move the component in the liquid means (hydrodynamics).

The inner part of the component is composed of a set of openings that form structured compartments (210, 310, 410, 510) so as to provide specific conditions favorable for developing biomass. The openings have an inner clearance that follows a ratio ranging between 1 and 10 mm, preferably at least 3.75 mm.

The inner structure forms a network composed of a series of inner rings (300, 400, 500) which form a set of openings and respective channels (210, 310, 320, 410, 510).

The elements of the inner rings (300, 400, 500) as well as the supporting means (100, 101) itself also have shapes and angulations resulting from technical researches, wherein such angulations provide greater protection to the biofilm and thus greater resistance potential to the reaction means.

The outer channels (210) are essentially triangular and formed between the outer ring (200) and the first inner ring (300), comprising a larger wall (216) domed and of concentric radius with the outer ring (200), flanked by two smaller curves (211, 215) adjacent to two straight smaller walls (212, 214) and which meet in a larger curve (213), completing a closed outline.

The first inner channels (310, 320) are essentially triangular and formed between the first inner ring (300) and the second inner ring (400), comprising a simple shape (310) and a complex shape (320).

The simple shape (310) comprises a straight larger wall (312) passing to a first curve (313), then to a smaller wall (314), the other curve (315), to a median wall (316), ending in another curve (311), completing a closed contour.

The complex shape (320) comprises a straight larger wall (324) passing to a first curve (325) with double corrugation, then to a smaller wall (326), the other curve (321), to a median wall (322), ending in another curve (323), completing a closed contour.

The second inner channels (410) are polygonal and formed between the second inner ring (400) and the third inner ring (500), comprising a first curve (411) adjacent to a straight first wall (412), an inflection (413) and a second wall (414) which in turn adjacent to a lower curve (415) with a concave section (416) adjacent to another lower curve (415), leading to a new second wall (414), a new inflection (413) and finally in a new first wall (412), completing a closed contour.

The third and last inner channel (510) between the third inner ring (500) and the geometric center of the supporting means (100), is polygonal and provided with sides (511) and vertices (512), preferably octagonally-shaped.

An alternative three-dimensional form of the supporting means (100) is an oblique supporting means (101), the walls and channels of which are oblique, having dimensions similar to the model described above, having an angle (α) ranging between 1° and 40°, being preferably 15°, in which the remaining features are unchanged.

It should be noted that the optimal use of the supporting means (100, 101) of the utility model in biological reactors comprises a mixture of elements, the angle (α) of which then ranges between 0° and 20° and the height (H) measurements of which range according to the corresponding hypotenuse or diameter (D).

This mixture of flat and oblique parts favors the development of different types of biomass in the biological reactor. The oblique components (101) have less resistance (friction) with the aqueous means and thus greater speed and shear potential, favoring the development of microorganisms with accelerated metabolism, such as heterotrophic bacteria (responsible for decomposing organic matter). On the other hand, the flat components (100) have a lower displacement speed and thus a less frequent shearing, increasing the conditions for the development of bacteria with less accelerated metabolism, such as autotrophic bacteria (responsible for means nitrification).

The techniques used when developing the supporting means (100, 101) aimed at the constructive balance of the component, providing significant gains in biomass performance, consequently in relation to energy consumption as well, contravening with the notion that MBBR (Moving Bed Biofilm Reactor)/IFAS (Integrated Fixed-Film Activated Sludge) systems consume high levels of energy. The specific density and unique shape make the interaction with the liquid means more accurate, without the need to inject larger air flows to obtain good conditions for moving the components.

The mixed use of flat and oblique components directly impacts the hydrodynamic effect in the reactor, increasing the shear effects between the components, as the friction with the aqueous means is reduced. The dimensions were also developed considering operational aspects, reducing the frequency of cleaning retention devices and facilitating the operational routine of treatment systems.

The rings (200, 300, 400, 500) and channels (210, 310, 320, 410, 520) were sized based on field data, providing balance between a shape that favors greater surface area while allowing for oxygen to reach every region of the component, also preventing the formation of anaerobic zones (zones without air which cause a reduction in treatment levels).

The network formed by rings (200, 300, 400, 500) and channels (210, 310, 320, 410, 520) allows for a significant increase in the biomass concentration capacity, due to the high surface area obtained and maintained in balance with the other constructive aspects of the component, favoring a uniform and quality biofilm.

Its construction provides greater protection to the biofilm, simultaneously with an opening of sections designed to obtain the maximum transportation of oxygen in every area of the component. The result is longer biomass retention time and superior sludge quality.

Furthermore, in addition to reducing friction with the aqueous means, the use of a supporting means set of the utility model optimizes the operational routines of wastewater treatment plants, reducing possible hydraulic and maintenance effects on aeration systems (less hydraulic loading and less maintenance on the media retention devices).

As for the material of the supporting means, a compound with controlled density is used, being the closest to the density of water, so as to allow for the best possible interaction with the liquid means, avoiding excess flotation or sedimentation of the component, aspects with a direct impact on the movement of suspended components and indirect impact on the energy consumption and the development of active biomass inside the reactor.

Preferred materials are plastic materials such as polypropylene, polyethylene, high density polyethylene and further equivalents, with specific densities ranging from 0.95 to 1 g/cm³, preferably between 0.98 g/cm³ to 0.99 g/cm³.

### FINAL REMARKS

It is clear that the measures and ratios between measurements described for the present structural arrangement may vary according to the dimensioning of supporting means (100, 101).

Exhaustive practical tests, however, have shown that these dimensions and their ratios are highly efficient and effective in safety and convenience provided by the supporting means (100).

Furthermore, the structural arrangement of the present utility model and the aforementioned measurements and rations thereof are highly reliable and reproducible.

### CONCLUSION

As can be inferred from the description above, the structural arrangement according to the present utility model overcomes prior-art solutions, being an object of practical use, perfectly prone to industrial application, which presents a novel arrangement, involving non-obviousness and resulting in functional improvement in use thereof.

1. A supporting means for wastewater treatment plants, characterized in that it comprises:
(i) An outer ring;
(ii) A first inner ring;
(iii) A second inner ring; and
(iv) A third inner ring;
wherein the outer ring is a cylindrical element of diameter (d), height (h), and thickness (e), that has an annular cross section and that delimits the network structure formed therein composed of the inner rings that form a set of openings and respective channels.

2. The supporting means for wastewater treatment plants, according to claim 1, characterized in that the ratio between diameter (d) and height (h) is between 1 and 5 times.

3. The supporting means for wastewater treatment plants, according to claim 1, characterized in that it comprises an oblique supporting means, having oblique walls and channels, with an angle (α) between 1° and 40°, forming a mixture of elements with the supporting means (100), the angle (α) of which is between 0° and 20° and the height (H) measurements of which ranges according to the corresponding hypotenuse or diameter (D).

4. The supporting means for wastewater treatment plants, according to claim 2, characterized in that the ratio between diameter (d) and height (h) ranges between 1 and 5 times.

5. The supporting means for wastewater treatment plants, according to claim 3, characterized in that the angle (α) between the oblique walls and channels is 15°.
